SEQUENCE LISTING (4 sequences)

SEQ ID NO: 1 - protein, 17 aa, Homo sapiens
CSIPPEVKFNKPFVYLI

SEQ ID NO: 2 - protein, 28 aa, Homo sapiens
CFLEAIPMSIPPEVKFNKPFVFLIIHRD

SEQ ID NO: 3 - protein, 5 aa, Homo sapiens
FVFLI

SEQ ID NO: 4 - protein, 5 aa, Homo sapiens
FVYLI

(19) United States
(12) Patent Application Publication
(10) Pub. No.: US 2004/0152653 A1
(43) Pub. Date: Aug. 5, 2004

(54) Enhanced delivery via serpin enzyme complex receptor

(75) Inventors: Assem Ziady, Cleveland, OH (US); Pamela B. Davis, Cleveland, OH (US); Thomas W. Ferkol JR., Cleveland, OH (US); Alfred Malouf, Cleveland, OH (US)

Correspondence Address: BANNER & WITCOFF, 1001 G STREET N W, SUITE 1100, WASHINGTON, DC, 20001, US

(73) Assignee: Case Western Reserve University, Cleveland, OH (US)

(21) Appl. No.: 10/703,206

(22) Filed: Nov. 7, 2003

Related U.S. Application Data
(63) Continuation of application No. PCT/US00/20545, filed on Jul. 28, 2000.
(60) Provisional application No. 60/145,970, filed on Jul. 29, 1999.

(51) Int. Cl. A61K 48/00

(52) U.S. Cl. 514/44

(57) ABSTRACT

Serpin enzyme complex receptors are used as targets for therapeutic drugs in the lungs and brain tissue. Any lung or brain disease and any therapeutic drug can be targeted to the lung or brain by use of ligands which specifically bind to the receptors. Complexes for delivery may include proteins, pharmacological agents, or nucleic acids, as well as carrier molecules, and ligands for the receptors. The ligands can be coupled directly to the therapeutic agent or to a carrier molecule which binds to the therapeutic agent.

This application claims the benefit of provisional application 06/145,970 filed Jul. 29, 1999, the disclosure of which is expresssly incorporated herein.

### TECHNICAL FIELD OF THE INVENTION

This invention is related to therapeutic methods for treating lung and brain diseases.

### BACKGROUND OF THE INVENTION

The serine protease inhibitor (serpin) enzyme complex receptor (SecR) is found on a variety of cell types, including hepatoma cells, mononuclear phagocytes, neutrophil cell lines, intestinal epithelial cell lines, mouse fibroblast cell lines, neuronal cell lines, and glial cell lines. This receptor binds to a region of serine protease inhibitors which is exposed by the proteolytic digestion of the serpin by its enzyme ligand with formation of a serpin/serine protease complex (Enghild, et al., 1994, J. Biol. Chem. 269:20159-20166; Perlmutter et al. 1990 J. Biol. Chem 265:16713-16716; Perlmutter et al. 1990 Proc. Natl. Acad. Sci. USA 87:3753-3757; Kahalil et al. 1994 Brain Res. 651:227-235; Joslin et al. 1991 J. Biol. Chem. 266:11282-11288; Joslin et al. 1993 J. Biol. Chem. 268:1886-1893.) Following binding, the serpin-enzyme complex is internalized and routed to the lysosomes for degradation. Synthetic peptides, based on sequence on amino acids 359-374 of α1-antiprotease, bind in a specific and saturable fashion to the receptor on HepG2 cells and mediate a functional response. The receptor also binds amyloid-βpeptide, substance P, and bombesin.

Peptides C105Y (CSIPPEVKFNKPFVYLI) (SEQ ID NO: 1) and C1315 (CFLEAIPMSIPPEVKFNKPFVFLIIHRD) (SEQ ID NO: 2) are two peptides which each contain the pentapeptide binding domain FV(F/Y)LI (SEQ ID NO: 3) necessary for binding to SecR.

Although there are certain agents available which have a beneficial effect on lung and brain diseases, their effects have been less than optimal. There is a continuing need in the art for new methods for increasing access to the airway epithelium and neurons to overcome barriers to effective treatment.

### SUMMARY OF THE INVENTION

It is an object of the present invention to provide methods of delivering therapeutic agents to airway epithelium of mammals.

It is another object of the present invention to provide methods of delivering therapeutic agents to brain tissue.

These and other objects of the invention are achieved by providing a method for delivering a therapeutic agent to airway epithelium of a mammal.

A therapeutic complex is administered to the airway epithelium via its luminal surface. The complex comprises a ligand for serpin enzyme complex receptor (SecR) and a therapeutic agent for treating lung disease.

According to another embodiment of the invention a method is provided for delivering nucleic acids to airway epithelium of a mammal. A nucleic acid complex is administered to the airway epithelium via its luminal surface. The complex comprises a ligand for serpin enzyme complex receptor (SecR), a carrier molecule, and a nucleic acid encoding a therapeutic agent for treating lung disease.

According to yet another embodiment of the invention a method is provided for delivering CFTR-encoding nucleic acids to the airway epithelium. A CFTR-encoding nucleic acid complex is administered to the luminal surface of the airway epithelium of a CF patient. The complex comprises a ligand for SecR coupled to a carrier molecule.

In yet another embodiment of the invention a method is provided for delivering a pharmacologic agent to brain tissue of a mammal. A pharmacologic complex is injected directly into the brain tissue. The complex comprises a ligand for serpin enzyme complex receptor (SecR) and a pharmacologic agent.

In still another embodiment of the invention a method is provided for delivering nucleic acids to brain tissue of a mammal. A nucleic acid complex is directly injected into the brain tissue. The complex comprises a ligand for serpin enzyme complex receptor (SecR), a carrier molecule, and a nucleic acid encoding a pharmacologic agent. The nucleic acid is expressed in the brain tissue.

Still another embodiment of the invention provides a use of a pharmacologic agent and a ligand for serpin enzyme complex receptor (SecR) in the preparation of a pharmacologic complex to be administered to airway epithelium via its luminal surface.

Still another embodiment of the invention provides a use of a nucleic acid encoding a pharmacologic agent and a ligand for serpin enzyme complex receptor (SecR) in the preparation of a pharmacologic complex to be administered to airway epithelium via its luminal surface.

Still another embodiment of the invention provides a use of a pharmacologic agent and a ligand for serpin enzyme complex receptor (SecR) in the preparation of a pharmacologic complex to be administered by direct injection to the brain.

Still another embodiment of the invention provides a use of a nucleic acid encoding a pharmacologic agent, a carrier molecule, and a ligand for serpin enzyme complex receptor (SecR) in the preparation of a pharmacologic complex to be administered by direct injection to the brain.

Still another embodiment of the invention provides a device for delivering a phamacologic complex to airway epithelium via its luminal surface, comprising a pharmacologic complex which comprises a pharmacologic agent and a ligand for SecR.

Still another embodiment of the invention provides a device for delivering a phamacologic complex to airway epithelium via its luminal surface, comprising a pharmacologic complex which comprises a nucleic acid encoding a pharmacologic agent and a ligand for SecR.

Still another embodiment of the invention provides a composition comprising a pharmacologic complex for delivery to airway epithelium via its luminal surface, said pharmacologic complex comprising a pharmacologic agent and a ligand for SecR.

Still another embodiment of the invention provides a composition comprising a pharmacologic complex for delivery by direct injection to brain, said pharmacologic complex comprising a pharmacologic agent and a ligand for SecR.

Still another embodiment of the invention provides a composition comprising a pharmacologic complex for delivery to airway epithelium via its luminal surface, said pharmacologic complex comprising a nucleic acid encoding a pharmacologic agent and a ligand for SecR.

Still another embodiment of the invention provides a composition comprising a pharmacologic complex for delivery by direct injection to the brain, said pharmacologic complex comprising a nucleic acid encoding a pharmacologic agent and a ligand for SecR.

Still another embodiment of the invention provides a use of a pharmacologic agent and a ligand for SecR in the preparation of a medicament for delivery to airway epithelium via its luminal surface for the treatment of lung disease.

Still another embodiment of the invention provides a use of a pharmacologic agent and a ligand for SecR in the preparation of a medicament for delivery by direct injection to the brain for the treatment of bacterial infection.

Still another embodiment of the invention provides a use of a pharmacologic agent and a ligand for SecR in the preparation of a medicament for delivery by direct injection to the brain for the treatment of viral infection.

Still another embodiment of the invention provides a use of a pharmacologic agent and a ligand for SecR in the preparation of a medicament for delivery by direct injection to the brain for the treatment of Alzheimer's disease.

Still another embodiment of the invention provides a use of a pharmacologic agent and a ligand for SecR in the preparation of a medicament for delivery by direct injection to the brain for the treatment of Parkinson's disease.

Still another embodiment of the invention provides a use of a pharmacologic agent and a ligand for SecR in the preparation of a medicament for delivery by direct injection to the brain for the treatment of a tumor.

Still another embodiment of the invention provides a use of a nucleic acid encoding a pharmacologic agent and a ligand for SecR in the preparation of a medicament for delivery to airway epithelium via its luminal surface for the treatment of lung disease.

Still another embodiment of the invention provides a use of a nucleic acid encoding a pharmacologic agent and a ligand for SecR in the preparation of a medicament for delivery by direct injection to the brain for the treatment of bacterial infection.

Still another embodiment of the invention provides a use of a nucleic acid encoding a pharmacologic agent and a ligand for SecR in the preparation of a medicament for delivery by direct injection to the brain for the treatment of viral infection.

Still another embodiment of the invention provides a use of a nucleic acid encoding a pharmacologic agent and a ligand for SecR in the preparation of a medicament for delivery by direct injection to the brain for the treatment of Alzheimer's disease.

Still another embodiment of the invention provides a use of a nucleic acid encoding a pharmacologic agent and a ligand for SecR in the preparation of a medicament for delivery by direct injection to the brain for the treatment of Parkinson's disease.

Still another embodiment of the invention provides a use of a nucleic acid encoding a pharmacologic agent and a ligand for SecR in the preparation of a medicament for delivery by direct injection to the brain for the treatment of a tumor.

Still another embodiment of the invention provides a use of a pharmacologic complex which comprises a pharmacologic agent and a ligand for SecR as a vehicle for the delivery of said pharmacologic agent to airway epithelium via its luminal surface.

Still another embodiment of the invention provides a use of a pharmacologic complex which comprises a nucleic acid encoding a pharmacologic agent, a carrier molecule, and a ligand for SecR as a vehicle for the delivery of said pharmacologic agent to airway epithelium via its luminal surface.

Still another embodiment of the invention provides a use of a pharmacologic complex which comprises a pharmacologic agent and a ligand for SecR as a vehicle for the delivery of said pharmacologic agent by direct injection to the brain.

Thus the present invention provides methods for treating lung disease by direct administration to the luminal surface of the airways and the apical surface of the epithelial cells. It also provides methods for treating brain disorders by targeting neuronal cells to enhance a therapeutic index.

### DETAILED DESCRIPTION

It is a discovery of the inventors that ligands which bind to SecR can be used to target therapeutic agents to the luminal surface of the lung, i.e., to the apical surface of the epithelial cells. Similarly, such ligands can be used to target neurons in the brain tissue. The use of the ligand enhances the therapeutic value of the agents, presumably because more of it is actually taken up by the target cells.

One of the uses of this unexpected targeting ability is for Cystic Fibrosis therapy using SecR-directed complexes applied from the luminal surface of the airway. Drugs such as 4-phenylbutyrate can be administered or polynucleotides encoding all or a portion of CFTR can be delivered to the surface of the airway by this means. Similarly, drugs can be administered to the brain for treating such neurological conditions as Parkinson's disease, Alzheimer's disease, and infections of neurons, whether bacterial or viral.

Complexes for delivery may or may not contain nucleic acids. Nucleic acids may be in the forms of liposomes, viruses, plasmids, compacted with proteins, or any other form suitable for delivery to cells. For example, one could envision attaching a SecR ligand to adenovirus and thereby markedly improving luminal access of the adenovirus to the airway epithelium. Similarly, this could be applied to AAV or retroviruses or lentiviruses. SecR ligands can also be incorporated into liposomes, such as by coupling to a component of the liposome. SecR ligands can also be directly coupled to a pharmacological agent.

Nucleic acids which can be used include DNA, RNA, DNA-RNA hybrids, and modified nucleic acids which contain nucleotide analogues which may improve the activity, stability, or uptake of the nucleic acids. The nucleic acids can be expected to have one or more biological effects on the cells which take them up. These include hybridization to complementary messenger RNA and inhibition of its translation, expression of the nucleic acid to form mRNA and/or protein, replication of the nucleic acid, homologous recombination to correct genetic errors, and integration of the nucleic acid.

Other lung disorders that one can treat by accessing the luminal surface of the airway via SecR include severe asthma, severe necrotizing pneumonia, α1-antitrypsin deficiency, chronic obstructive pulmonary disease, and bronchogenic carcinomas. Suitable therapeutic agents include, but are not limited to proteins or the genes encoding them. Suitable agents for treating these severe lung diseases include blockers of cytokine receptors, such as interleukin-4 or -13 receptors, anti-imflammatory cytokines, α1-antitrypsin, inhibitors of mucin synthesis, mucin antisense, inhibitors of mucin secretion, protease inhibitors, and anti-tumor agents.

Any ligand known in the art to bind to the serpin enzyme complex can be used. These include ligands comprising FV(F/Y)LI (SEQ ID NO: 3), such as peptides C105Y and C1315. Any receptor which binds these ligands can be targeted.

Carrier molecules according to the present invention are typically substances which are biocompatible and relatively inert immunologically. These include proteins, polypeptides, lipids, liposomes, etc. Particularly preferred is a polymer having a polylysine backbone. A cysteine or other moieties may be attached to the polylysine.

Modes of administration which may be used to access the luminal surface of the airway epithelium include instillation into the nose, inhalation, delivery of an aerosol via the nose or the mouth, delivery via fluorocarbon liquid ventilation of the airways, etc. Any means known in the art for reaching the airways can be used. Devices such as inhalers, and nebulizers can be used, some of which may contain a predetermined dose of pharmacological complex. Similarly, administration to the apical surface of an oriented sheet of epithelial cells in vitro can also be used. For delivery to brain tissue cells, direct injection may be guided by direct vision or stereotactic control. Such direct injection bypasses the blood-brain barrier.

Nucleic acid and other pharmacologic complexes may be delivered to subjects according to the present invention for the purpose of screening for agents which enhance nucleic acid or pharmacologic agent transfer to cells or subsequent biological effects of the nucleic acids or pharmacologic agents. Agents which can be screened include any test compounds or substances, whether natural products or synthetic, which can be administered to the subject. Libraries or mixtures of compounds can be tested. The compounds or substances may be those for which a pharmaceutical effect is previously known or unknown. The compounds or substances may be delivered before, after, or concomitantly with the nucleic acid or pharmacologic complexes. They may be administered separately or in admixture with the nucleic acid or pharmacologic complexes. Integration of delivered DNA or other pharmacologic agent can be monitored by any means known in the art. For example, Southern blotting of the delivered DNA can be performed. A change in the size of the fragments of the delivered nucleic acid indicates integration. Replication of the delivered nucleic acid can be monitored inter alia by monitoring incorporation of labeled nucleotides combined with hybridization to a probe for the delivered nucleic acids. Expression of the nucleic acid can be monitored by detecting production of RNA which hybridizes to the delivered nucleic acid or by detecting protein encoded by the delivered nucleic acid. A protein can be detected immunologically or by activity, for example. Recombination can be determined by sequencing, or hybridization or observation of restoration of function. Thus the delivery of the nucleic acid or pharmacologic complexes according to the present invention provides an excellent system for screening agents for their ability to promote delivery, integration, hybridization, expression, replication or integration in an animal, preferably a mammal, more preferably a human.

The above disclosure generally describes the present invention. A more complete understanding can be obtained by reference to the following specific examples which are provided herein for purposes of illustration only, and are not intended to limit the scope of the invention.

### EXAMPLE 1

We have demonstrated that the gene encoding CFTR (the cystic fibrosis transmembrane conductance regulator protein) can be successfully transferred to the nasal epithelium of cystic fibrosis (CF) mice by direct instillation in the nasal cavity of complexes consisting of the C105Y ligand directed at the serpin-enzyme complex (SEC) receptor, coupled to polylysine, condensed with plasmid DNA, and expressed at a level which is detectable by electrophysiologic measurements.

The Experiments

CF knockout mice, which do not express CFTR, underwent measurement of nasal potential difference (PD) and were confirmed to have nasal potential difference measurements characteristics of cystic fibrosis—that is, no (or negative) response to superfusion with solution containing low chloride concentrations plus isoproterenol. This maneuver increases the electrochemical gradient for chloride and increases intracellular cAMP, which should activate the CFTR chloride channel. If chloride is secreted, there will be a change in the electrical potential across the epithelium of the mouse. Each of the mice used for the experiment had characteristic CF nasal PD trace—that is, a slightly negative response to these maneuvers.

At least two days following the initial PD measurements, mice were treated with one of the following complexes:

C105Y-polylysine-plasmid DNA containing CFTR.

C105Y-polylysine-plasmid DNA containing lac Z.

Polylysine-plasmid DNA containing CFTR.

Complexes (containing 1.5 ug DNA, compacted with equal-charge amounts of polylysine) were applied to the nasal epithelium of anesthetized CF mice in 30 ul volume of a solution ˜1.1 M NaCl. Complexes were applied slowly, and the mice did sniff some of the material into the lung. The mice were allowed to recover from anesthesia and return to their cages. Four days later nasal PD measurements were repeated. For the 3 animals treated with polylysine-plasmid DNA with CFTR, or the 3 treated with C105Y-polylysine-plasmid DNA with lac Z, there were no changes in nasal PD response to superfusion with low chloride plus isoproterenol containing solutions. For the four CF mice who received C105Y-polylysine-plasmid DNA with CFTR, one had no change, two had traces that were slightly positive, and one had a nearly normal trace.

| | Pretreatment PD | Day 4 PD | Change from |
|---|---|---|---|
| | (Δ with low | (Δ with low | pretreatment |
| Complex | C1-/iso), mV | C1-/iso), mV | mV |
| C105Y/polyK/DNA- | −3.8 ± 2.3 | 1.4 ± 2.6 | 5.2 |
| CFTR | | | |
| C105Y/polyK/DNA- | −2.0 ± 1.3 | −3.7 ± 1.8 | −1.7 |
| lacZ | | | |
| polyK/DNA-CFTR | −4.3 ± 3.2 | −4.4 ± 0.9 | −0.1 |

Normal ΔPD with low Cl—, iso is about 14 mV.

We interpret these data to indicate that the SEC receptor can facilitate uptake and expression of compacted DNA into the nasal epithelium via the apical surface. Moreover the uptake and expression is sufficient to provide at least partial electrophysiologic correction at four days. This result does not occur from nonspecific uptake, because the complexes containing no ligand show no electrophysiologic correction. This result does not occur from nonspecific changes in the cell physiology due to accessing the cells via the SEC receptor, because complexes made with C105Y but containing the lac Z gene did not produce electrophysiologic correction. Although there are no good data to use as a reference point to assess the meaning of the degree of correction, the animal who achieved nearly normal electrophysiology would be expected to have therapeutic benefit, and reversal of the negative trend in CF patients (to less negative or slightly positive) has been touted as a therapeutic triumph for 4-phenylbutyrate, a drug purported to improve processing of the AF508 mutant of CFTR. The amount of DNA administered is modest, and no dose-response or time course data are available.

Confirmation of gene delivery was obtained from the animals given the lacZ gene. These animals show extensive blue staining of the nasal epithelium, larynx, and spotty staining of the tracheal and bronchial epithelium, confirming that foreign genes are delivered to the appropriate cells and expressed.

### EXAMPLE 2

We pursued the ability to transfer genes into airway epithelial cell via SEC-R in vitro models. Two human airway epithelial cell line, 9HTEo—(which does not form tight junctions) and 16HBEEo-cells (which do form tight junctions) can be transfected with SEC-R directed complexes, though these experiments were done with cells grown on plastic and not polarized. These cells never achieve the high levels of expression we see in human hepatoma HuH7 cells, nor is the duration of expression as long. To further pursue the observations, we grew human tracheal epithelial cells in primary cultures to confluence on filters, and demonstrated that they formed a polarized monolayer. Using fluorescein-tagged C105Y peptide, we demonstrated that there was binding of the peptide to the apical surface of airway epithelial cells. Moreover, we were able to effect transfer of a reporter gene, green fluorescent protein, to primary cultures of polarized human airway epithelial cells using SEC-R directed complexes applied to the apical surface. Interestingly, in vitro, C1315 ligand was as efficacious as C105Y. It was these data that encouraged us to test the ability to correct the CF mouse in vivo. These data also indicate that this system accesses human airway epithelial cells as well as mouse airway epithelial cells.

### EXAMPLE 3

We have demonstrated that genes encoding either green fluorescent protein or bacterial β-galactosidase can be expressed in neurons in rat brain slices following direct microinjection. About 1-10 picoliters of a solution of gene transfer complex containing 1 ug plasmid DNA per 20 microliters (about 0.5-5 picograms DNA) was injected into the hippocampal area of rat brain slices about 200 microns in thickness. For green fluorescent protein, the sections were examined by fluorescent microscopy for several days thereafter, and for β-galactosidase the sections were fixed and stained with X-gal solution for 3 hours, then examined by light microscopy. Control samples were treated with the same genes complexed with polyethlyeneimine or with polylysine with no ligand. For both of the controls, gene transfer occurred, but only to cells with the morphology of glial cells. For the complexes containing the SecR ligand, cells with the morphology of neurons were transfected as well. We interpret these data to show that SecR directed complexes can deliver foreign genes to neurons when they are presented by direct injection.

1. A method for delivering a pharmacoligic agent to airway epithelium of a mammal, comprising the step of:
administering a pharmacologic complex to the airway epithelium via its luminal surface, wherein the complex comprises a ligand for serpin enzyme complex receptor (SecR) and a pharmacologic agent.

2. A method for delivering nucleic acids to airway epithelium of a mammal, comprising the step of:
administering a nucleic acid complex to the airway epithelium via its luminal surface, wherein the complex comprises a ligand for serpin enzyme complex receptor (SecR), a carrier molecule, and a nucleic acid encoding a pharmacologic agent, whereby the nucleic acid is expressed in the airway epithelium.

3. A method for delivering CFTR-encoding nucleic acids to the airway epithelium, comprising:
administering a CFTR-encoding nucleic acid complex to the luminal surface of the airway epithelium of a CF patient wherein the complex comprises a ligand for SecR coupled to a carrier molecule, whereby CFTR is expressed in the airway epithelium.

4. A method for delivering a pharmacologic agent to brain tissue of a mammal, comprising the step of:
directly injecting into the brain a pharmacologic complex, wherein the complex comprises a ligand for serpin enzyme complex receptor (SecR) and a pharmacologic agent.

5. A method for delivering nucleic acids to brain tissue of a mammal, comprising the step of:
directly injecting a nucleic acid complex to the brain tissue, wherein the complex comprises a ligand for serpin enzyme complex receptor (SecR), a carrier molecule, and a nucleic acid encoding a pharmacologic agent, whereby the nucleic acid is expressed in the brain tissue.

6. Use of a pharmacologic agent and a ligand for serpin enzyme complex receptor (SecR) in the preparation of a pharmacologic complex to be administered to airway epithelium via its luminal surface.

7. Use of a nucleic acid encoding a pharmacologic agent and a ligand for serpin enzyme complex receptor (SecR) in the preparation of a pharmacologic complex to be administered to airway epithelium via its luminal surface.

8. Use of a pharmacologic agent and a ligand for serpin enzyme complex receptor (SecR) in the preparation of a pharmacologic complex to be administered by direct injection to the brain.

9. Use of a nucleic acid encoding a pharmacologic agent, a carrier molecule, and a ligand for serpin enzyme complex receptor (SecR) in the preparation of a pharmacologic complex to be administered by direct injection to the brain.

10. A device for delivering a phamacologic complex to airway epithelium via its luminal surface, comprising a pharmacologic complex which comprises a pharmacologic agent and a ligand for SecR.

11. A device for delivering a phamacologic complex to airway epithelium via its luminal surface, comprising a pharmacologic complex which comprises a nucleic acid encoding a pharmacologic agent and a ligand for SecR.

12. A composition comprising a pharmacologic complex for delivery to airway epithelium via its luminal surface, said pharmacologic complex comprising a pharmacologic agent and a ligand for SecR.

13. A composition comprising a pharmacologic complex for delivery by direct injection to brain, said pharmacologic complex comprising a pharmacologic agent and a ligand for SecR.

14. A composition comprising a pharmacologic complex for delivery to airway epithelium via its luminal surface, said pharmacologic complex comprising a nucleic acid encoding a pharmacologic agent and a ligand for SecR.

15. A composition comprising a pharmacologic complex for delivery by direct injection to the brain, said pharmacologic complex comprising a nucleic acid encoding a pharmacologic agent and a ligand for SecR.

16. The use of a pharmacologic agent and a ligand for SecR in the preparation of a medicament for delivery to airway epithelium via its luminal surface for the treatment of lung disease.

17. The use of a pharmacologic agent and a ligand for SecR in the preparation of a medicament for delivery by direct injection to the brain for the treatment of bacterial infection.

18. The use of a pharmacologic agent and a ligand for SecR in the preparation of a medicament for delivery by direct injection to the brain for the treatment of viral infection.

19. The use of a pharmacologic agent and a ligand for SecR in the preparation of a medicament for delivery by direct injection to the brain for the treatment of Alzheimer's disease.

20. The use of a pharmacologic agent and a ligand for SecR in the preparation of a medicament for delivery by direct injection to the brain for the treatment of Parkinson's disease.

21. The use of a pharmacologic agent and a ligand for SecR in the preparation of a medicament for delivery by direct injection to the brain for the treatment of a tumor.

22. The use of a nucleic acid encoding a pharmacologic agent and a ligand for SecR in the preparation of a medicament for delivery to airway epithelium via its luminal surface for the treatment of lung disease.

23. The use of a nucleic acid encoding a pharmacologic agent and a ligand for SecR in the preparation of a medicament for delivery by direct injection to the brain for the treatment of bacterial infection.

24. The use of a nucleic acid encoding a pharmacologic agent and a ligand for SecR in the preparation of a medicament for delivery by direct injection to the brain for the treatment of viral infection.

25. The use of a nucleic acid encoding a pharmacologic agent and a ligand for SecR in the preparation of a medicament for delivery by direct injection to the brain for the treatment of Alzheimer's disease.

26. The use of a nucleic acid encoding a pharmacologic agent and a ligand for SecR in the preparation of a medicament for delivery by direct injection to the brain for the treatment of Parkinson's disease.

27. The use of a nucleic acid encoding a pharmacologic agent and a ligand for SecR in the preparation of a medicament for delivery by direct injection to the brain for the treatment of a tumor.

28. Use of a pharmacologic complex which comprises a pharmacologic agent and a ligand for SecR as a vehicle for the delivery of said pharmacologic agent to airway epithelium via its luminal surface.

29. Use of a pharmacologic complex which comprises a nucleic acid encoding a pharmacologic agent, a carrier molecule, and a ligand for SecR as a vehicle for the delivery of said pharmacologic agent to airway epithelium via its luminal surface.

30. Use of a pharmacologic complex which comprises a pharmacologic agent and a ligand for SecR as a vehicle for the delivery of said pharmacologic agent by direct injection to the brain.

31. Use of a pharmacologic complex which comprises a nucleic acid encoding a pharmacologic agent, a carrier molecule, and a ligand for SecR as a vehicle for the delivery by direct injection to the brain.

32. The method of claim 1 or 4 wherein the complex further comprises a carrier molecule.

33. The method of claim 2, 5, or 32 wherein the carrier molecule is coupled to the ligand for SecR.

34. The method of claim 33 wherein the carrier molecule is a lipid.

35. The method of claim 33 wherein the pharmacologic complex is a liposome.

36. The method or use of claim 1, 6, or 16 wherein the pharmacologic agent is 4-phenylbutyrate.

37. The method or use of claim 1, 6, or 16 wherein the pharmacologic agent is α1-antitrypsin.

38. The method or use of claim 1, 6, or 16 wherein the pharmacologic agent is a phosphodiesterase inhibitor.

39. The method, composition or use of claim 2, 7, 14, 22, or 29 wherein the pharmacologic agent is cystic fibrosis transmembrane conductance regulator protein (CFTR).

40. The method, composition or use of claim 2, 7, 14, 22, or 29 wherein the pharmacologic agent is a cytokine receptor blocker.

41. The method, composition or use of claim 40 wherein the pharmacologic agent is an IL-4 receptor blocker.

42. The method, composition or use of claim 40 wherein the pharmacologic agent is an IL-13 receptor blocker.

43. The method, composition or use of claim 2, 7, 14, 22, or 29 wherein the pharmacologic agent is an anti-inflammatory cytokine.

44. The method, composition or use of claim 2, 7, 14, 22, or 29 wherein the pharmacologic agent is α1-antitrypsin.

45. The method, composition or use of claim 2, 7, 14, 22, or 29 wherein the pharmacologic agent is a protease inhibitor.

46. The method, composition or use of claim 2, 7, 14, 22, or 29 wherein the pharmacologic agent is an inhibitor of mucin synthesis.

47. The method, composition or use of claim 2, 7, 14, 22, or 29 wherein the pharmacologic agent is an inhibitor of mucin secretion.

48. The method, use, or composition of claim 2, 3, or 5 wherein the carrier molecule is polylysine.

49. The method, use, or composition of claim 2, 5, 7, 14, 22, or 29 wherein the nucleic acid complex is a virus.

50. The method, composition or use of claim 49 wherein the virus is adenovirus.

51. The method, composition or use of claim 49 wherein the virus is adeno-associated virus.

52. The method, composition or use of claim 49 wherein the virus is a retrovirus.

53. The method, composition or use of claim 49 wherein the virus is a lentivirus.

54. The method of claim 1 or 2 wherein the mammal has cystic fibrosis.

55. The method of claim 1 or 2 wherein the mammal has asthma.

56. The method of claim 1 or 2 wherein the mammal has severe necrotizing pneumonia.

57. The method of claim 1 or 2 wherein the mammal has α1-antitrypsin deficiency.

58. The method of claim 1 or 2 wherein the mammal has a chronic obstructive pulmonary disease.

59. The method of claim 1 or 2 wherein the mammal has a bronchogenic carcinoma.

60. The method of claim 1 or 4 wherein the pharmacologic agent is an anti-tumor agent.

61. The method of claim 1, 2, 3, 4, or 5 wherein the ligand is C105Y.

62. The method of claim 1, 2, 3, 4, or 5 wherein the ligand is C1315.

63. The method of claim 1, 2, 3, 4, or 5 wherein the step of administering is via the nose.

64. The method of claim 2, 3, or 5 wherein the carrier molecule is cysteine-polylysine.

65. The method of any of claims 1-30 wherein the ligand comprises FV(F/Y)LI (SEQ ID NO: 3).

66. The method of claim 4 or 5 wherein the pharmacologic agent is useful for treating brain disorders.

67. The method of claim 4 or 5 wherein the pharmacologic agent is a protease inhibitor.

68. The method of claim 4 or 5 wherein the pharmacologic agent is an antibiotic.

69. The method of claim 4 or 5 wherein the pharmacologic agent is an anti-viral agent.

70. The method of claim 4 or 5 wherein the mammal has Alzheimer's disease.

71. The method of claim 4 or 5 wherein the mammal has Parkinson's disease.

72. The method of claim 4 or 5 wherein the mammal has an intraneuronal infection.

73. The device of claim 10 or 11 which is a nebulizer.

74. The device of claim 10 or 11 which is an inhaler.

75. The device of claim 10 or 11 which delivers pre-determined doses.
